(19) United States
(12) Patent Application Publication
(10) Pub. No.: US 2011/0040142 A1
(43) Pub. Date: Feb. 17, 2011

(54) VACUUM ERECTION CONTROL SYSTEM

(75) Inventors: JAY J. EUM, Irvine, CA (US); Paul Lapine, Laguna Niguel, CA (US); Chris Kinnison, Mansfield, TX (US)

Correspondence Address: LAWRENCE N. GINSBERG, 21 SAN ANTONIO, NEWPORT BEACH, CA, 92660-9112, US

(73) Assignee: FIRMA MEDICAL HOLDINGS, LLC, Mansfield, TX (US)

(21) Appl. No.: 12/842,384

(22) Filed: Jul. 23, 2010

Related U.S. Application Data
(60) Provisional application No. 61/234,573, filed on Aug. 17, 2009.

(51) Int. Cl. A61F 5/41

(52) U.S. Cl. 600/38

(57) ABSTRACT

A vacuum erection control system for erectile dysfunction treatment. The vacuum erection control system includes a vacuum erection device, including a vacuum chamber and a thin elastic seal element. The vacuum chamber has a distal end for operable connection to a vacuum generating means, an open proximal end, an inside surface and an outside surface. The thin elastic seal element is formed of a stretchable material having a first seal end and a second seal end. The first seal end is stretched for removable sealing placement on the proximal end of the vacuum chamber. The second seal end has a central orifice forming a penile seal for the sealable introduction of a penis. The stretchable material is substantially non-permeable to air and expandable to at least twice its original area without losing this non-permeable characteristic. During operation the first seal end is secured on the proximal end of the vacuum chamber, the operator's penis is introduced through the penile seal central opening and a vacuum is produced by the vacuum generating means operatively connectable to the vacuum chamber. As an erection is enhanced the penis advances toward the distal end of the vacuum chamber and commensurately the penile seal advances toward the base of the penis.

### BACKGROUND OF THE INVENTION

1. Field of the Invention

The present invention relates generally to vacuum erection devices and therapy for erectile dysfunction treatment and augmenting male potency.

2. Description of the Related Art

Current VED Technology/Systems

Vacuum erection devices (VEDS) are a mechanical means of achieving an erection. They rely on the principle of creating a negative pressure around the penis which draws in blood. When the penis is erect a ring is slipped off the pump onto the penile base, thus trapping the engorged blood and maintaining the erection.

Prescribed for men diagnosed with erectile dysfunction (ED) there are a number of pre-requisites to their use. Firstly, the penis must be of reasonable length for ease of application. Secondly, and perhaps most importantly, the patient and his partner should be highly motivated and well instructed in the technique. Most urologists believe VED therapy to be a useful and effective treatment modality for ED, but patients will often report that:

1) VEDs are too mechanical.

2) The ring is not tight enough.

3) The ring is so tight that it is difficult to remove. (Soderdahl D W, Thrasher J B, Hansberry K L. Intracavernosal Drug Induced Erection Therapy Versus External Vacuum Devices in the Treatment of Erectile Dysfunction. Br J Urol 1997;79:952-957.)

So, while VEDs are effective, studies indicate that more than 90% of men using these devices are able to get an erection that is satisfactory for sexual intercourse, patient satisfaction with the device and the quality of the erections is less impressive. Some studies have reported satisfaction rates ranging from 50% to 70%. However, other studies report that only 26% to 30% of men are satisfied with the erections they receive from a vacuum device. Most men who stop using the vacuum device do so for a variety of reasons; including inconvenience and interruption of foreplay (the man has to take a brief “time-out” to use the device to get an erection). (Web MD: http://www.webmd.com/erectile-dysfunction/vacuum-devices-for-erection-problems, as downloaded

The main cause of the patient complaints and the subsequent low patient compliance is related to the current VED technology, cost and the convoluted method of using the systems to achieve and sustain an erection. Several vendors today publish 30+page instruction books on how to assemble, disassemble and clean their systems. As a result, today's VED technology is far less satisfying than it is capable of being.

Creating an Erection With Current VED Systems

To create an erection patients start by attaching a vacuum pump onto one end of a cylinder creating a closed cylinder end. Once the pump is attached to the cylinder, patients must than check the open cylinder end to see if the rubber insert is needed to reduce the cylinder diameter. This would be required if the patient's penis is significantly thinner in girth than the diameter of the cylinder. Next, the patient must apply a thick water soluble lubricating gel in three places: 1) two inches inside the open cylinder, 2) the rim on the cylinder which meets the body; and, 3) the entire head of the penis. The gel is used to help create an air tight seal between the body and the chamber. Once a healthy amount of lubricate is applied to the open end of the cylinder the patient places the cylinder over his penis and then presses the cylinder tightly against his pelvic/abdomen to provide an airtight seal. Any air leak will prevent an erection from accruing. Patients are also encouraged to trim their pubic hair around the base of the penis to help improve the airtight seal and minimize hair being sucked into the cylinder. With the cylinder held tightly against the body with one hand the patient must now operate the vacuum pump either by squeezing the handle on the hand pump or by pressing the power button on the battery pump. The pump allows the air to escape within the cylinder creating a negative pressure and thereby facilitating the patient's blood to flow into the penis. During the pumping process the patient must operate the pump gradually and intermittently. Pumping fast and/or in a constant action will typically result in a poor erection or no erection and can promote bruising and discomfort. Because the VED cylinder must to be held tightly against the body, often the patient's skin around the penis and pubic hair is sucked into the open cylinder causing bruising, mild pain or discomfort.

Key patient complaints associated with achieving an erection with current VED systems includes:

1) Time and incontinence of assembly, disassembly and cleaning the cylinder and rubber insert.

2) The time, expense, mess and incontinence of applying lubricant on the cylinder and penis.

3) Lack of an erection due to an air leak.

4) Lack of an erection due to fast or constant pumping.

5) Pulling of patient's pelvic tissue and pubic hair creating bruising, pain and inconvenience.

### Sustaining an Erection

To sustain an erection patients apply a constriction ring at the base of their penis. The constriction ring inhibits the blood flow away from the penis thus sustaining the erection. To work effectively the constriction ring must be the right diameter. If it is too large then the erection will not be maintained. If it is too small the patient will experience significant discomfort and even mild pain. To apply the constriction ring patients must first preliminarily install the ring on the vacuum cylinder. To accomplish this, some VED systems have special constriction ring tools wherein the patient uses the constriction ring applicator to stretch the constriction ring over the base of the cylinder. Other systems require the patient to use lubricate and brute force to stretch and load the ring. Once the ring is stretched and loaded into the cylinder the force is used to slip the ring off the cylinder and onto the base of the penis. Once the constriction ring is in place and the erection is being sustained, the vacuum is released and the chamber is removed from the erect penis. Following intercourse the patient must than remove the constriction ring. The rings should not be kept on longer than 30 minutes. To remove the ring the patient must grasp the outer loops of the ring (note not all rings have outer loops) and pull outward and hold for 10-15 seconds while the penis loses some firmness. Afterward the ring can be rolled off. It is important that the right size ring is used otherwise removing the constriction ring can be difficult.

Key patient complaints associated with sustaining an erection with current VED systems includes:

1) Loading the constriction ring onto the cylinder is to complex and requires too much force.

2) Forcing the constriction ring off the cylinder and onto the base of the penis is painful.

3) The constriction ring is too loose and the erection cannot be sustained.

4) The constriction ring is too tight and painful.

5) Removing the ring is clumsy, and in some cases painful.

History of VED systems

VED's have been prescribed by physicians to treat ED for well over 20 years. ED, which affects millions of men to various degrees, is defined as the persistent inability to attain or maintain penile erection sufficient for sexual intercourse. The majority of cases have an organic etiology that typically involves underlying conditions related to blood flow, nerve damage or sever psychosocial disorder. Causes include: prostate and colon cancer, diabetes, chronic lung disease, coronary artery disease, arthritis, hypertension, nerve disorder, quadriplegia, paraplegia, and severe depression. Treatments for ED include Oral Medication, Injection Therapy, Hormone Therapy, Penile Implants, Urethral Suppositories and VED's. Vacuum erection devices are designed to produce artificial erections by creating a vacuum that draws blood into the penis. Once the blood is in the penis, a special rubber band is placed at the base of the penis, and the vacuum device is removed. This leaves the man with a penis engorged with blood and erect. The band needs to be removed immediately at the end of intercourse. In addition to organic ED, which is often a chronic and life long condition, approximately 70% of patients who undergo prostate cancer treatment experience some transient or temporary (6 to 18 months) loss of normal sexual function. For patients with transient ED following prostate cancer treatment, many physicians prescribe a comprehensive rehabilitation therapy often combining oral medications (such as PDE5 blockers) and daily VED use to mimic nocturnal erections which research shows helps to avoid atrophy, penile shortening and fibrosis along with helping to promote an early return to sexual function. The clinical theory is that proactively treating patients postoperatively for presumed nerve damage stimulates nerve recovery and possibly reduces the degree of irreversible damage. The underlying hypothesis is that the artificial induction of erections shortly after surgery facilitates tissue oxygenation, reducing cavernosal fibrosis in the absence of nocturnal erections, potentially increasing the likelihood of preserving ED. VED's, because of their ability to draw blood into the penis regardless of nerve disturbance, have become the centerpiece of penile rehabilitation protocols.

Presently manufactured VED's comprise a vacuum chamber with an open end serving as an entrance, a closed end connected to a vacuum pump and a constriction ring placed on the vacuum chamber close to its open end. To achieve an erection the penis is inserted into the open end of the vacuum chamber which is pressed to abdomen to provide an airtight seal. Then the vacuum is generated in the chamber with the manually or electrically (battery) operated vacuum pump. The partial vacuum inside the chamber causes the blood flow into the penis thus producing an erection. To sustain the erection the constriction ring preliminary installed on the vacuum chamber is forced to slip off onto the root of the penis. Being placed on the root of the penis the constriction ring inhibits the blood flow from the penis thus sustaining erection. After this, the vacuum is released, and the chamber is removed from the erect penis.

Numerous studies and surveys show that vacuum constriction therapy can help patients to improve sexual satisfaction, decrease psychiatric symptomatology, and, increase self-esteem. (Roy Witherington, “Suction Device Therapy in the Management of Erectile Impotence”, Urologic Clinics of North America v. 15, No. 1, February 1988); D. E. Price et al. “The Management of Impotence in Diabetic Men by Vacuum Tumescence Therapy”, Diabetic Medicine, 1991; 964-967). W. Meinhardt et al. “The Negative Pressure Device for Erectile Disorders: When Does It Fail?”; Journal of Urology, v. 149, p.p. 1285.

There are numerous other patents that have issued that have involved erectile dysfunction. U.S. Pat. No. 6,248,059, entitled “Powered External Vacuum Appliance for the Treatment of Impotence,” issued to Gamper et al, discloses a motorized or manual pump that attaches to one end of a cylinder with attaching rings to the other end as necessary to reduce diameter.

U.S. Pat. No. 4,378,008, entitled “Erection Aid Device,” issued to Osbon Sr., discloses a device which includes an elongated vacuum cylinder which receives the male organ and which is connected by means of a flexible conduit to a vacuum source by which the interior of the cylinder may be evacuated to cause the organ to become erect and distended. A manually operated valve is connected in the flexible conduit which may be easily closed to maintain the partial vacuum and organ erect in the cylinder while an elastic band carried adjacent the open end of the cylinder is fitted over the base of the organ whereafter the valve may be opened to relieve the vacuum and remove the vacuum cylinder with the erection being captured.

U.S. Pat. No. 4,856,498, entitled “Vacuum Generating and Constriction Apparatus for Augmenting Male Potency,” issued to Osbon, discloses a vacuum chamber, manual vacuum pump and a hose connecting the chamber and pump. U.S. Pat. No. 5,344,389, entitled “Combinational Seal and Constricting Device,” issued to Walsdorf et al., and U.S. Pat. No. 6,705,987, entitled “Penile Seal and Constriction Ring” issued to Anderson et al disclose a ring and seal combination which is inserted to the flaccid penis first.

U.S. Pat. No. 6,036,635, entitled “Erection Control System,” issued to Y. Altshuler, discloses a penis-shaped vacuum chamber adapted for unnoticeable operation under a user's cloth. A removably placed penile seal and a baffle are adjustable to the user's penile girth and provide attachment so that the vacuum chamber hangs on the user's penis without additional support. A ribbon-shaped constriction device is wound with multiple turns over each other to form a cylindrical ring, retained by the belt for exerting a prearranged inward radial pressure. A transferring device with the pulling loop dislodges the constriction device together with the removably placed penile seal and baffle onto the erect penis with simultaneous release of vacuum without additional mechanisms. The removably placed penile seal, the baffle, and the belt are formed from segments of a condom.

### SUMMARY OF THE INVENTION

In a broad aspect, the present invention is a vacuum erection control system for erectile dysfunction treatment. The vacuum erection control system includes a vacuum erection device, including a vacuum chamber and a thin elastic seal element. The vacuum chamber has a distal end for operable connection to a vacuum generating means, an open proximal end, an inside surface and an outside surface. The thin elastic seal element is formed of a stretchable material having a first seal end and a second seal end. The first seal end is stretched for removable sealing placement on the proximal end of the vacuum chamber. The second seal end has a central orifice forming a penile seal for the sealable introduction of a penis. The stretchable material is substantially non-permeable to air and expandable to at least twice its original area without losing this non-permeable characteristic. During operation the first seal end is secured on the proximal end of the vacuum chamber, the operator's penis is introduced through the penile seal central opening and a vacuum is produced by the vacuum generating means operatively connectable to the vacuum chamber. As an erection is enhanced the penis advances toward the distal end of the vacuum chamber and commensurately the penile seal advances toward the base of the penis.

A separable, adjustable constriction clamp is placed around the penis between the base of the penis and the penile seal when a desired erection is achieved, for maintaining the erection upon removal of the vacuum erection device.

The present invention addresses key patient complaints associated with existing VED technology/systems. The present invention provides patients a simpler, more effective and less costly VED option. In addition to being an integrated system the component technology comprising the present invention is backward compatible to present assignee, Firma Medical's, existing VED systems that are sold under the trademarks Ultra™ and Classic™ and can be easily used to help improve the functionally, clinical effectiveness and operating costs of the current Firma Medical devices.

### BRIEF DESCRIPTION OF THE DRAWINGS

FIG. 1 is a perspective illustration of the vacuum erection control system of the present invention, shown assembled.

FIG. 2 is an exploded perspective illustration of the vacuum erection control system of the present invention, including the constriction clamp for use therewith.

FIG. 3 is perspective illustration, partially in cross-section, taken along line 3-3 of FIG. 1.

FIG. 4 is a cross-sectional view of the proximal end of the vacuum erection control system showing the seal element positioned in an inverted fashion prior to operation.

FIG. 5 is a cross-sectional view of the vacuum erection control system showing introduction of the penis in a first step of operation.

FIG. 6 illustrates the advancement of the penis toward the distal end of the vacuum chamber.

FIG. 7 illustrates the application of the constriction clamp.

FIG. 8 shows the final step in which the vacuum chamber is removed.

### DETAILED DESCRIPTION OF THE INVENTION

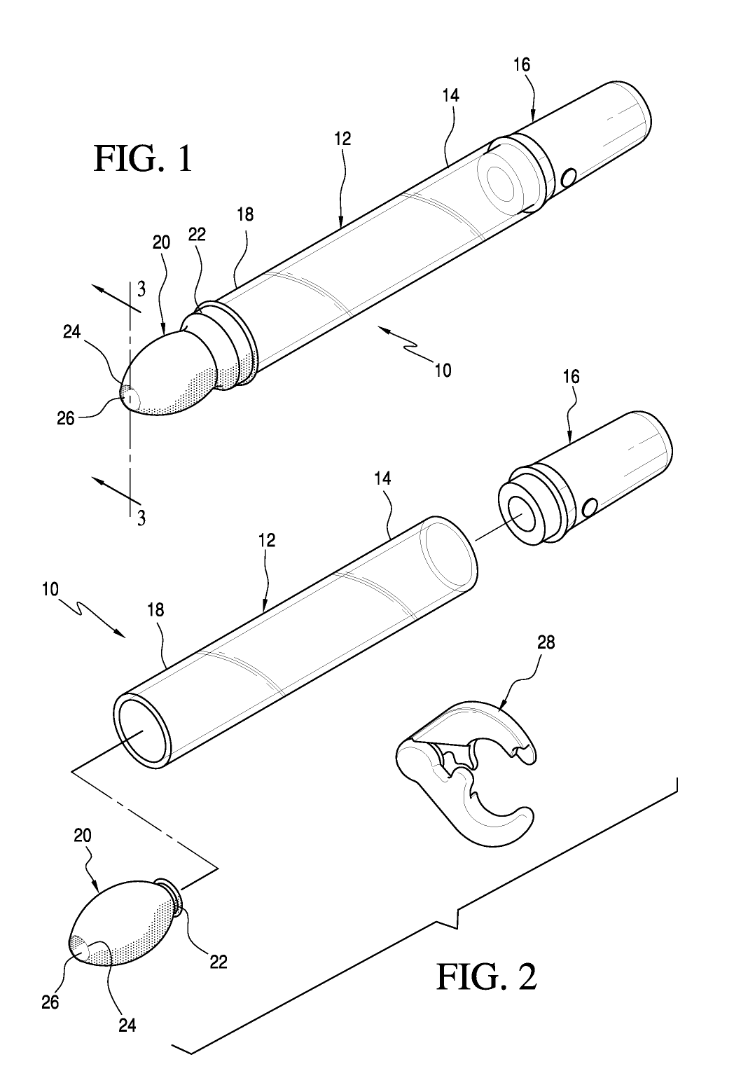

Referring now to the drawings and the characters of reference marked thereon, FIGS. 1 and 2 illustrate the vacuum erection control system of the present invention, designated generally as 10. The vacuum erection control system 10 includes a vacuum chamber, designated generally as 12, having a distal end 14 for operable connection to a vacuum generating means 16, an open proximal end 18, an inside surface, and an outside surface. A thin elastic seal element, designated generally as 20, includes a first seal end 22 for removable sealing placement on the proximal end of the vacuum chamber 12. A second seal end 24 has a central orifice 26 forming a penile seal for the sealable introduction of a penis.

The vacuum chamber 12 is tubular and is preferably formed of plastic which may be marked as to where the vacuum pump should be placed. The proximal (i.e., penis end) of the vacuum chamber 12 may optionally have a grooved ridge or a flared end to help hold the elastic seal element 20 onto the cylinder. The chamber (i.e. cylinder) 12 may also have measurement markings along the side of the chamber 12 allowing patients to measure the length of their penis. Also, optionally, the chamber 12 may be constructed to have a slightly smaller opening on the proximal end for better accommodating the seal element 20.

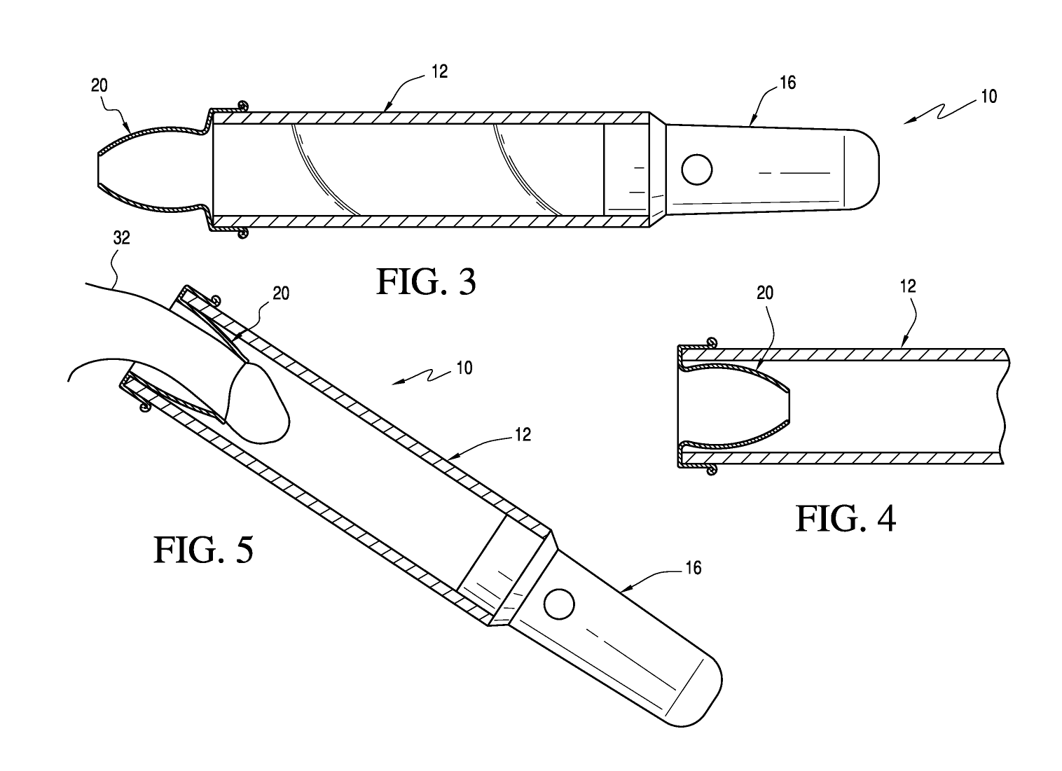

The seal element 20 is formed of stretchable material that is substantially non-permeable to air and expandable to at least twice its original area without losing this non-permeable characteristic. The seal is preferably formed of thin latex rubber material. Silicon seals are available for patients with latex allergies. The seal element 20 has an unstretched length, as defined by the distance between the first seal end 22 and the second seal end 24, in a range of about 0.5 inches and 3.0 inches. It is about 2.5 inches wide. The first seal end 22 is preferably rolled over itself to facilitate application to and removal from the vacuum chamber 12, as can be seen clearly in FIG. 3. The seal element is preferably less than about 50/1000 inches (50 mil) thick.

The vacuum generating means 16 is preferably a motorized (typically battery powered) operated vacuum pump. An alternative vacuum pump is a hand pump. The pump 16 preferably has an intermittent pump cycle. It also preferably has a timer alert. The intermittent pump cycle starts and stops the vacuum cycle automatically based on the timer setup. It helps to eliminate the problem of poor erection or no erection resulting when patients pump to fast and/or in a constant action. Furthermore, the intermittent pump cycle helps to reduce bruising and discomfort. The timer alert feature uses a button that when pressed beeps after a set time alerting the patient that the set amount of time has elapsed (typically 10 minutes). The timer is helpful for patients during practice/training sessions and for use during penile rehabilitation sessions.

As will be explained below in more detail, a constriction clamp, designated generally as 28, is placeable around the penis between the base of the penis and the penile seal when a desired erection is achieved, for maintaining the erection upon removal of the vacuum erection device 10. The constriction clamp 28 may be, for example, a separable, adjustable constriction clamp, as shown in FIG. 2, which preferably utilizes a double ratchet system wherein patients squeeze the clamp to the desired level of closure. As used herein, the term “separable” refers to the two ends of the clamp 28. The clamp 28 is shown in an open, separated position in FIG. 2. The closed clamp 28 traps the blood in the penis similar to currently used constriction rings by squeezing the veins on the top and sides of the erect penis. Although not shown in this figure, the clamp 28 may have a quick-&-easy release lever, for safety and convenience. Patients may use the lever to easily adjust or remove the clamp 28. The clamp 28 replaces the rubber constriction rings and the constriction ring applicator of the prior art and it is reusable. The clamp 28 provides patients an option with better control, more convenience, less pain and potentially lower cost. Additionally, the clamp 28 is very simple to apply and highly intuitive to patients.

Referring now to FIG. 4, prior to operation the seal element 20 is positioned in an inverted fashion within the cylinder 12. As shown in FIG. 5, the penis 32 is then introduced through the penile seal central opening 26 and a vacuum is produced by the vacuum generating means 16 operatively connectable to the vacuum chamber 12. Since the seal element 20 closes off the end of the cylinder 12 the vacuum is created much easier than with prior art devices, thereby helping to ensure better erection success. Likewise, because the seal closes off the end of the cylinder 12 the patient no longer needs the lubricant or the rubber insert; and, no longer needs to press the cylinder up against his pelvic abdomen. As a result patient avoids the inconvenience and cost of applying lubricant. Additionally, the skin around the penis and pubic hair is kept from being sucked into the open cylinder 12, improving the patient's experience and reducing tissue bruising and discomfort.

As the vacuum is created within the cylinder 12, and the penis 32 expands in length and girth, the seal 20 stretches allowing the penis 32 to pass though the opening. As the penis 32 passes though the seal opening 26 the patient's penile skin receives a positive sensation. The sensation acts as an external simulate helping to enhance the patient's erection and possibly promote increased blood flow. The seal 20 is held in place by the grooved ridge (or a flair end) on the cylinder 12. By using the seal 20 patients no longer need the lubricant or the rubber insert. As a result the seal 20 provides many patient benefits, including: more effective erections, positive sensational feedback, lower cost, less mess, no rubber insert, no pressure against the body, no hair and tissue disruption, and less bruising. One advantage of the seal lies in the fact that the material is very thin and highly stretchable, thereby providing a snug yet comfortable fit around the penis as it expands with the vacuum chamber. The “umbrella” effect, shown in the Figures, results in minimizing the contact area between the penis 32 and the seal 20. These effects create a unique patient experience compared to current VED options. It is more satisfying and effective.

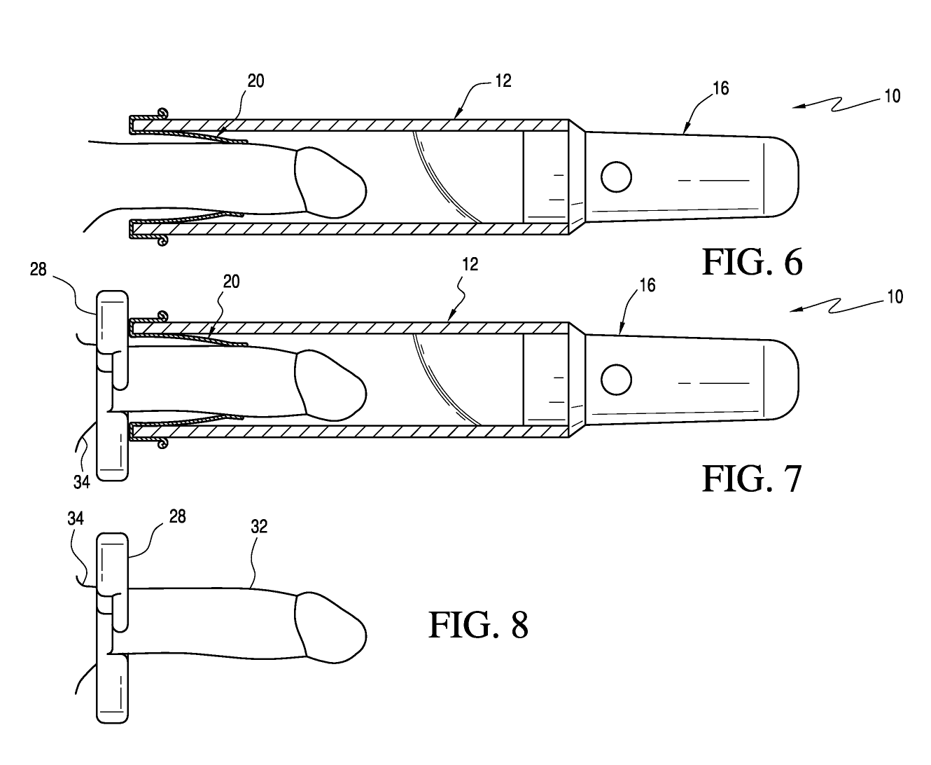

FIG. 6 shows that as an erection is enhanced the penis 32 advances toward the distal end 14 of the vacuum chamber 12 and commensurately the penile seal 20 advances toward the base 34 of the penis 32.

FIG. 7 illustrates the application of the constriction clamp 28 after the desired erection is achieved. The clamp 28 provides patients a unique option providing better control, more convenience, less pain and potentially lower cost than current VED systems.

Finally, as shown in FIG. 8, the vacuum chamber 12 is removed.

After use the constriction clamp is simply unclamped.

Other embodiments and configurations may be devised without departing from the spirit of the invention and the scope of the appended claims.

1. A vacuum erection control system for erectile dysfunction treatment, comprising a vacuum erection device, said vacuum erection device comprising:
a) a vacuum chamber having a distal end for operable connection to a vacuum generating means, an open proximal end, an inside surface and an outside surface; and,
b) a thin elastic seal element formed of a stretchable material having a first seal end and a second seal end, said first seal end being stretched for removable sealing placement on said proximal end of said vacuum chamber, said second seal end having a central orifice forming a penile seal for the sealable introduction of a penis, said stretchable material being substantially non-permeable to air and expandable to at least twice its original area without losing this non-permeable characteristic,
wherein during operation said first seal end is secured on said proximal end of said vacuum chamber, the operator's penis is introduced through said penile seal central opening and a vacuum is produced by the vacuum generating means operatively connectable to said vacuum chamber,
wherein as an erection is enhanced the penis advances toward said distal end of said vacuum chamber and commensurately the penile seal advances toward the base of the penis.

2. The vacuum erection control system of claim 1, wherein said vacuum erection device further comprises a vacuum generating means operatively connected to said vacuum chamber for producing a vacuum in said vacuum chamber.

3. The vacuum erection control system of claim 1, further comprising a separable, adjustable constriction clamp placeable around said penis between the base of the penis and said penile seal when a desired erection is achieved, for maintaining the erection upon removal of said vacuum erection device.

4. The vacuum erection control system of claim 1, wherein said vacuum chamber is tubular.

5. The vacuum erection control system of claim 1, wherein said thin elastic seal element is formed of latex rubber or silicon.

6. The vacuum erection control system of claim 1, wherein said thin elastic seal element has an unstretched length, as defined by the distance between said first seal end and said second seal end, in a range of about 0.5 inches and 3.0 inches.

7. The vacuum erection control system of claim 1, wherein said first end of said thin elastic seal element is rolled to facilitate application to and removal from said vacuum chamber.

8. A method for treating erectile dysfunction, comprising the steps of:
a) providing a vacuum chamber having a distal end for operable connection to a vacuum generating means, an open proximal end, an inside surface and an outside surface;
b) providing a thin elastic seal element formed of a stretchable material having a first seal end and a second seal end, said first seal end being stretched for removable sealing placement on said proximal end of said vacuum chamber, said second seal end having a central orifice forming a penile seal for the sealable introduction of a penis, said stretchable material being substantially non-permeable to air and expandable to at least twice its original area without losing this non-permeable characteristic;
c) positioning said thin elastic seal element on said proximal end of said vacuum chamber;
d) generating an erection by introducing the operator's penis through said orifice of said penile seal and applying vacuum to said vacuum chamber, wherein as an erection is enhanced the penis advances toward said distal end of said vacuum chamber and commensurately the penile seal advances toward the base of the penis;
e) positioning a separable, adjustable constriction clamp over the penis adjacent to said proximal end of said vacuum chamber and said penile seal; and,
f) clamping said constriction clamp after the desired erection is achieved.

9. A vacuum erection control system for erectile dysfunction treatment, comprising
a) a vacuum erection device, comprising:
i. a vacuum chamber having a distal end for operable connection to a vacuum generating means, an open proximal end, an inside surface and an outside surface;
ii. a thin elastic seal element formed of a stretchable material having a first seal end and a second seal end, said first seal end being stretched for removable sealing placement on said proximal end of said vacuum chamber, said second seal end having a central orifice forming a penile seal for the sealable introduction of a penis, said stretchable material being substantially non-permeable to air and expandable to at least twice its original area without losing this non-permeable characteristic; and,
iii. a vacuum generating means operatively connected to said vacuum chamber for producing a vacuum in said vacuum chamber; and,
b) a separable, adjustable constriction clamp placeable around said penis between the base of the penis and said penile seal when a desired erection is achieved, for maintaining the erection upon removal of said vacuum erection device,
wherein during operation said first seal end is secured on said proximal end of said vacuum chamber, the operator's penis is introduced through said penile seal central opening and a vacuum is produced by the vacuum generating means operatively connectable to said vacuum chamber,
wherein as an erection is enhanced the penis advances toward said distal end of said vacuum chamber and commensurately the penile seal advances toward the base of the penis.
